(19) United States
(12) Patent Application Publication
(10) Pub. No.: US 2017/0349520 A1
(43) Pub. Date: Dec. 7, 2017

(54) SINGLY PROTECTED 2,2' -DIHYDROXYBIARYLS

(71) Applicants: Katrin Marie DYBALLA, Recklinghausen (DE); Robert FRANKE, Marl (DE); Dirk FRIDAG, Haltern am See (DE); Siegfried R. WALDVOGEL, Gau-Algesheim (DE); Bernd ELSLER, Bonn (DE); Anton WIEBE, Neuwied (DE)

(72) Inventors: Katrin Marie DYBALLA, Recklinghausen (DE); Robert FRANKE, Marl (DE); Dirk FRIDAG, Haltern am See (DE); Siegfried R. WALDVOGEL, Gau-Algesheim (DE); Bernd ELSLER, Bonn (DE); Anton WIEBE, Neuwied (DE)

(73) Assignee: EVONIK DEGUSSA GMBH, Essen (DE)

(21) Appl. No.: 15/508,244

(22) PCT Filed: Jul. 23, 2015
(86) PCT No.: PCT/EP2015/066815

§ 371 Date: Mar. 2, 2017

(30) Foreign Application Priority Data
Sep. 3, 2014 (DE) 102014217542.9
Apr. 22, 2015 (DE) 102015207280.0

(51) Int. Cl. C07C 43/23; C07F 7/08; C07C 317/18; C25B 3/10; C07C 69/78

(52) U.S. Cl. CPC C07C 43/23; C25B 3/10; C07C 69/78; C07C 317/18; C07F 7/0818

(57) ABSTRACT

Novel singly protected 2,2′-dihydroxybiaryls, electrochemical process for preparation of singly protected 2,2′-dihydroxybiaryls.

The invention relates to novel singly protected 2,2′-dihydroxybiaryls, and to an electrochemical process for preparation of singly protected 2,2′-dihydroxybiaryls.

The terms “phenols” and “naphthols” are used as generic terms in this application and therefore also encompass substituted phenols/naphthols.

Electrochemical processes for cross-coupling of phenols with singly O-protected phenol derivatives, beyond the O-methylated derivatives, are unknown to date.

The synthesis of the target structures was performable to date by the conventional cross-coupling of phenols and a subsequent unselective protection of a hydroxyl function (see: G. Sartori, R. Mass, F. Bigi, M. Grandi, J. Org. Chem., 1993, 58, 7271-7273).

A great disadvantage of the abovementioned methods for phenol cross-coupling is the need for dry solvents and for exclusion of air. Moreover, large amounts of oxidizing agents, some of which are toxic, are often used. The tolerance of functional groups is often restricted by the reagents used. Toxic by-products often occur during the reaction, and have to be removed from the desired product in a complex manner and disposed of at great cost.

Similarly costly and inconvenient is the subsequent demethylation of methyl-protected 2,2′-biphenols by means of strong Lewis acids, for example BBr₃, Al(III) halides, or NbCl₅. Very severe, electrophilic reaction conditions are required here.

An electrochemical process for coupling of phenols with arenes bearing methoxy groups is described by A. Kirste, B. Elsler, G. Schnakenburg, S. R. Waldvogel, in J. Am. Chem. Soc., 2012, 134, 3571-3576. Depending on the reaction regime, this process produces a large amount of homo-coupling product as a secondary component. In a further step, after the homo-coupling, a protecting group is introduced. The product yields are moderate, and secondary components are obtained, which have to be removed in a complex manner. Since this complex separation takes place at the end of the reaction, this means that the costly product of value (protected 2,2′-dihydroxybiaryls) is unavoidably lost as well, since every purification step inevitably also leads to minimization of the yield of the target product.

The subsequent introduction of a protecting group into the dihydroxybiaryl is marked by a poor selectivity in most cases. Removal of many by-products from the second synthesis step is the consequence. There has to date been no one-stage route to unsymmetric, partly protected dihydroxybiaryl derivatives. The cross-coupling of O-methylated phenol derivatives and subsequent electrophilic deprotection are likewise not promising. An additional factor is that tert-butyl groups, silyl substituents and iodine substituents are incompatible under the conditions mentioned, as a result of which the substrate range is highly restricted. An alternative nucleophilic deprotection, for example with thiolate or cyanide, in contrast, has great toxic risks. The safety measures that have to be observed would be increased considerably as a result.

The problem addressed by the invention was that of providing novel singly protected 2,2′-dihydroxybiaryls having novel structures compared to the 2,2′-dihydroxybiaryls known in the literature. In addition, a process by which the novel 2,2′-dihydroxybiaryls can be prepared in good yield was to be developed. More particularly, the process was to stand out advantageously from the preparation processes known from the prior art.

The object is achieved by a compound according to claim 1.

Compound having one of the general structures (I) to (IIb):

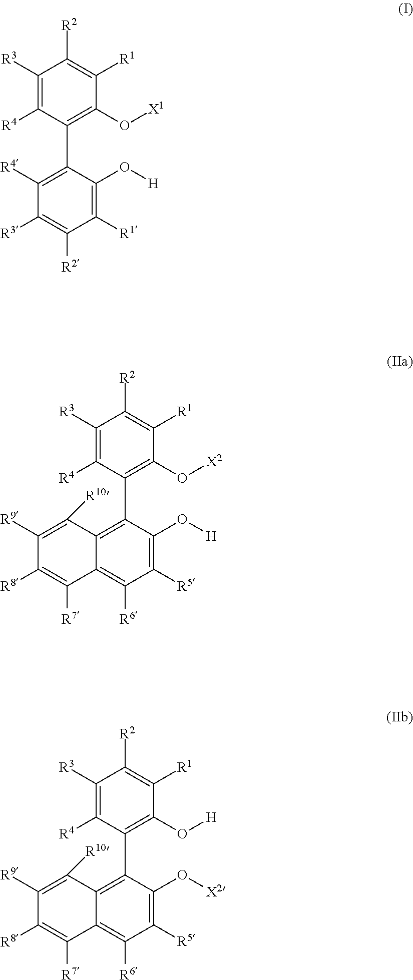

where
R¹, R², R³, R^{1′}, R^{2′}, R^{3′}, R^{4′} are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —CN, —N[(C₁-C₁₂)-alkyl]₂;
R^{5′}, R^{6′}, R^{7′}, R^{8′}, R^{9′}, R^{10′} are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —N[(C₁-C₁₂)-alkyl]₂;
where the alkyl and aryl groups mentioned may be substituted;
and, in the formula (I), the two radicals in at least one of the four following radical pairs are not the same radical: R¹ and R^{1′}, R² and R^{2′}, R³ and R^{3′}, R⁴ and R^{4′},
X² is selected from:
tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl, tetrahydropyranyl, -allyl ether, -benzyl, -p-methoxybenzyl, -3,4-dimethoxybenzyl, -aryl, -methoxymethyl,
X¹, X^{2′} is selected from:
tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl.

The feature “and the two radicals in at least one of the four following radical pairs are not the same radical: R¹ and R^{1′}, R² and R^{2′}, R³ and R^{3′}, R⁴ and R^{4′}” expresses the fact that this is an unsymmetric biaryl. The two aromatic systems cannot be reflected onto one another by a mirror plane lying between them. Not even if X¹ were to be H.

The following radical pairs are permitted, for example:

R¹≠R^{1′}, R²=R^{2′}, R³=R^{3′}, R⁴=R^{4′};
R¹=R^{1′}, R²=R^{2′}, R³≠R^{3′}, R⁴=R^{4′}.

But also radical pairs in which more than just one pair is not the same, for example:

R¹≠R^{1′}, R²=R^{2′}, R³≠R^{3′}, R⁴=R^{4′};
R¹≠R^{1′}, R²≠R^{2′}, R³≠R^{3′}, R⁴=R^{4′}.

The only case ruled out is that in which all four radical pairs are each the same radical in pairs:

R¹=R^{1′}, R²=R^{2′}, R³=R^{3′}, R⁴=R^{4′}.

This would be a symmetric biaryl.

—(C₁-C₁₂)-Alkyl and —O—(C₁-C₁₂)-alkyl may each be unsubstituted or substituted by one or more identical or different radicals selected from —(C₃-C₁₂)-cycloalkyl, —(C₃-C₁₂)-heterocycloalkyl, —(C₆-C₂₀)-aryl, fluorine, chlorine, cyano, formyl, acyl and alkoxycarbonyl.

—(C₆-C₂₀)-Aryl and —(C₆-C₂₀)-aryl-(C₆-C₂₀)-aryl- may each be unsubstituted or substituted by one or more identical or different radicals selected from:

—H, —(C₁-C₁₂)-alkyl, —O—(C₁-C₁₂)-alkyl, —O—(C₆-C₂₀)-aryl, —(C₆-C₂₀)-aryl, -halogen (such as Cl, F, Br, I), —COO—(C₁-C₁₂)-alkyl, —CONH—(C₁-C₁₂)-alkyl, —(C₆-C₂₀)-aryl-CON[(C₁-C₁₂)-alkyl]₂, —CO—(C₁-C₁₂)-alkyl, —CO—(C₆-C₂₀)-aryl, —COOH, —OH, —SO₃H, —SO₃Na, —NO₂, —CN, —N[(C₁-C₁₂)-alkyl]₂.

In the context of the invention, the expression “—(C₁-C₁₂)-alkyl” encompasses straight-chain and branched alkyl groups. Preferably, these groups are unsubstituted straight-chain or branched —(C₁-C₈)-alkyl groups and most preferably —(C₁-C₆)-alkyl groups. Examples of —(C₁-C₁₂)-alkyl groups are especially methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1,1,2-trimethylpropyl, 1,2,2-trimethylpropyl, 1-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The elucidations relating to the expression —(C₁-C₁₂)-alkyl also apply to the alkyl groups in —O—(C₁-C₁₂)-alkyl, i.e. in —(C₁-C₁₂)-alkoxy. Preferably, these groups are unsubstituted straight-chain or branched —(C₁-C₆)-alkoxy groups.

Substituted —(C₁-C₁₂)-alkyl groups and substituted —(C₁-C₁₂)-alkoxy groups may have one or more substituents, depending on their chain length. The substituents are preferably each independently selected from —(C₃-C₁₂)-cycloalkyl, —(C₃-C₁₂)-heterocycloalkyl, —(C₆-C₂₀)-aryl, fluorine, chlorine, cyano, formyl, acyl and alkoxycarbonyl.

The expression “—(C₃-C₁₂)-cycloalkyl”, in the context of the present invention, encompasses mono-, bi- or tricyclic hydrocarbyl radicals having 3 to 12, especially 5 to 12, carbon atoms. These include cyclopropyl, cyclobutyl, cyclopentyl, cyclohexyl, cycloheptyl, cyclooctyl, cyclododecyl, cyclopentadecyl, norbonyl and adamantyl.

One example of a substituted cycloalkyl would be menthyl.

The expression “—(C₃-C₁₂)-heterocycloalkyl groups”, in the context of the present invention, encompasses nonaromatic saturated or partly unsaturated cycloaliphatic groups having 3 to 12, especially 5 to 12, carbon atoms. The —(C₃-C₁₂)-heterocycloalkyl groups have preferably 3 to 8, more preferably 5 or 6, ring atoms. In the heterocycloalkyl groups, as opposed to the cycloalkyl groups, 1, 2, 3 or 4 of the ring carbon atoms are replaced by heteroatoms or heteroatom-containing groups. The heteroatoms or the heteroatom-containing groups are preferably selected from —O—, —S—, —N—, —N(═O)—, —C(═O)— and —S(═O)—. Examples of —(C₃-C₁₂)-heterocycloalkyl groups are tetrahydrothiophenyl, tetrahydrofuryl, tetrahydropyranyl and dioxanyl.

In the context of the present invention, the expression “—(C₆-C₂₀)-aryl and —(C₆-C₂₀)-aryl-(C₆-C₂₀)-aryl-” encompasses mono- or polycyclic aromatic hydrocarbyl radicals. These have 6 to 20 ring atoms, more preferably 6 to 14 ring atoms, especially 6 to 10 ring atoms. Aryl is preferably —(C₆-C₁₀)-aryl and —(C₆-C₁₀)-aryl-(C₆-C₁₀)-aryl-. Aryl is especially phenyl, naphthyl, indenyl, fluorenyl, anthracenyl, phenanthrenyl, naphthacenyl, chrysenyl, pyrenyl, coronenyl. More particularly, aryl is phenyl, naphthyl and anthracenyl.

Substituted —(C₆-C₂₀)-aryl groups and —(C₆-C₂₀)-aryl-(C₆-C₂₀)-aryl groups may have one or more (e.g. 1, 2, 3, 4 or 5) substituents, depending on the ring size. These substituents are preferably each independently selected from —H, —(C₁-C₁₂)-alkyl, —O—(C₁-C₁₂)-alkyl, —O—(C₆-C₂₀)-aryl, —(C₆-C₂₀)-aryl, -halogen (such as Cl, F, Br, I), —COO—(C₁-C₁₂)-alkyl, —CONH—(C₁-C₁₂)-alkyl, —(C₆-C₂₀)-aryl-CON[(C₁-C₁₂)-alkyl]₂, —CO—(C₁-C₁₂)-alkyl, —CO—(C₆-C₂₀)-aryl, —COOH, —OH, —SO₃H, —SO₃Na, —NO₂, —CN, —N[(C₁-C₁₂)-alkyl]₂.

Substituted —(C₆-C₂₀)-aryl groups and —(C₆-C₂₀)-aryl-(C₆-C₂₀)-aryl groups are preferably substituted —(C₆-C₁₀)-aryl groups and —(C₆-C₁₀)-aryl-(C₆-C₁₀)-aryl groups, especially substituted phenyl or substituted naphthyl or substituted anthracenyl. Substituted —(C₆-C₂₀)-aryl groups preferably bear one or more, for example 1, 2, 3, 4 or 5, substituents selected from —(C₁-C₁₂)-alkyl groups, —(C₁-C₁₂)-alkoxy groups.

The expression “halogens” encompasses Cl, F, Br, I, preferably Cl, Br, I.

In one embodiment, X² is selected from;

tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl.

In one embodiment, X¹ is selected from:

tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester.

In one embodiment, X² is selected from:

tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester.

In one embodiment, X^{2′} is selected from:

tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester.

In one embodiment, X¹ is selected from:

tert-butyl, -methylthioethyl, -trimethylsilyl, -triethylsilyl, -triisopropylsilyl, -tert-butyldimethylsilyl, -tert-butyldiphenylsilyl, -benzoic ester.

In one embodiment, X² is selected from:

tert-butyl, -methylthioethyl, -trimethylsilyl, -triethylsilyl, -triisopropylsilyl, -tert-butyldimethylsilyl, -tert-butyldiphenylsilyl, -benzoic ester.

In one embodiment, X^{2′} is selected from:

tert-butyl, -methylthioethyl, -trimethylsilyl, -triethylsilyl, -triisopropylsilyl, -tert-butyldimethylsilyl, -tert-butyldiphenylsilyl, -benzoic ester.

In one embodiment, X¹ is selected from:

-trimethylsilyl, -triethylsilyl, -triisopropylsilyl, -tert-butyldimethylsilyl, -tert-butyldiphenylsilyl.

In one embodiment, X² is selected from:

-trimethylsilyl, -triethylsilyl, -triisopropylsilyl, -tert-butyldimethylsilyl, -tert-butyldiphenylsilyl.

In one embodiment, X^{2′} is selected from:

-trimethylsilyl, -triethylsilyl, -triisopropylsilyl, -tert-butyldimethylsilyl, -tert-butyldiphenylsilyl.

In one embodiment, X¹ is selected from:

tert-butyl, -methylthioethyl, -acetyl, -pivaloyl, -benzoic ester.

In one embodiment, X² is selected from:

tert-butyl, -methylthioethyl, -acetyl, -pivaloyl, -benzoic ester.

In one embodiment, X^{2′} is selected from:

tert-butyl, -methylthioethyl, -acetyl, -pivaloyl, -benzoic ester.

In one embodiment, X¹ is selected from:

tert-butyl, -acetyl, -pivaloyl, -benzoic ester.

In one embodiment, X² is selected from:

tert-butyl, -acetyl, -pivaloyl, -benzoic ester.

In one embodiment, X^{2′} is selected from:

tert-butyl, -acetyl, -pivaloyl, -benzoic ester.

In one embodiment, X¹ is selected from:

-acetyl, -pivaloyl.

In one embodiment, X^{2′} is selected from:

-acetyl, -pivaloyl.

In one embodiment, X^{2′} is selected from;

-acetyl, -pivaloyl.

In one embodiment, R¹, R², R³, R⁴, R^{1′}, R^{2′}, R^{3′}, R^{4′} are selected from:

—H, —(C₁-C₁₂)-alkyl, —O—(C₁-C₁₂)-alkyl, —O—(C₆-C₂₀)-aryl, —S-alkyl, —S-aryl, halogen.

In one embodiment, R¹, R², R³, R⁴, R^{1′}, R^{2′}, R^{3′}, R^{4′} are selected from:

—H, —(C₁-C₁₂)-alkyl, —O—(C₁-C₁₂)-alkyl, —O—(C₆-C₂₀)-aryl.

In one embodiment, R^{5′}, R^{6′}, R^{7′}, R^{8′}, R^{9′}, R^{10′} are selected from:

—H, —(C₁-C₁₂)-alkyl, —O—(C₁-C₁₂)-alkyl, —(C₆-C₂₀)-aryl, —S-alkyl, —S-aryl, halogen.

In one embodiment, R^{5′}, R^{6′}, R^{7′}, R^{8′}, R^{9′}, R^{10′} are selected from:

—H, —(C₁-C₁₂)-alkyl, —O—(C₁-C₁₂)-alkyl, —O—(C₆-C₂₀)-aryl.

In one embodiment, R¹ and R^{1′} are not the same radical.

In one embodiment, R² and R^{2′} are not the same radical.

In one embodiment, R³ and R^{3′} are not the same radical.

In one embodiment, R⁴ and R^{4′} are not the same radical.

In one embodiment, R^{5′}, R^{6′}, R^{7′}, R^{8′}, R^{9′}, R^{10′} are the same radical.

In one embodiment, R^{1′}, R^{2′}, R^{3′}, R^{4′} are the same radical.

In one embodiment, R¹, R², R³, R⁴ are the same radical.

In one embodiment, the compound has the general structure (I).

In one embodiment, the compound has the general structure (IIa) or (IIb).

In one embodiment, the compound has the general structure (IIa).

In one embodiment, the compound has the general structure (IIb).

As well as the compounds, processes for preparation of 2,2′-dihydroxybiaryls are also claimed.

Process for preparing 2,2′-dihydroxybiaryls, comprising the process steps of:

a1) reacting a compound of the formula (IVa):

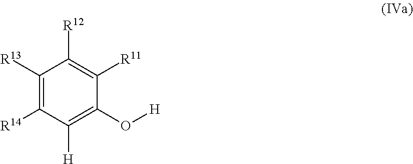

with X¹¹ to give (IVb)

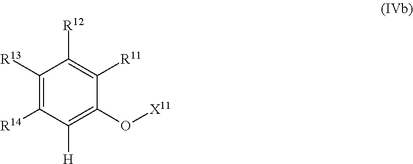

b1) electrochemically coupling:

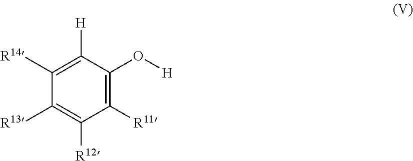

(IVb) with (V) to give (VI)
with use of the compound having the higher oxidation potential in excess:

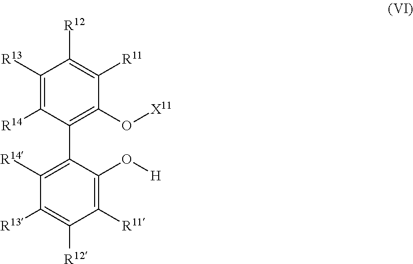

where
R¹¹, R¹², R¹³, R¹⁴, R^{11′}, R^{12′}, R^{13′}, R^{14′} are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —CN, —N[(C₁-C₁₂)-alkyl]₂;
where the alkyl and aryl groups mentioned may be substituted;
X¹¹ is selected from:
tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl, tetrahydropyranyl, -allyl ether, -benzyl, -p-methoxybenzyl, -3,4-dimethoxybenzyl, -aryl, -methoxymethyl.

Process for preparing 2,2′-dihydroxybiaryls, comprising the process steps of:

a2) reacting a compound of the formula (VIIa):

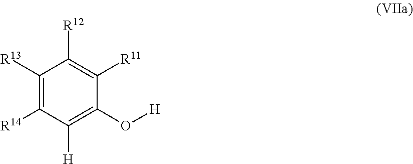

with X¹² to give (VIIb):

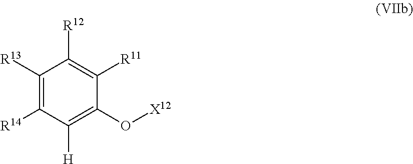

b2) electrochemically coupling:

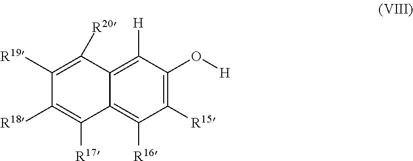

(VIIb) with (VIII) to give (IX)
with use of the compound having the higher oxidation potential in excess:

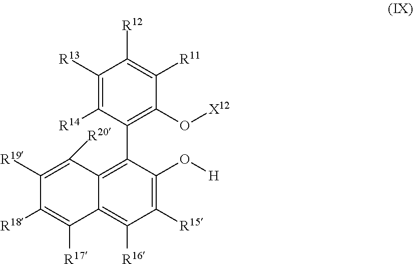

where
R¹¹, R¹², R¹³, R¹⁴ are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —CN, —N[(C₁-C₁₂)-alkyl]₂;
R^{15′}, R^{16′}, R^{17′}, R^{18′}, R^{19′}, R^{20′} are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —N[(C₁-C₁₂)-alkyl]₂;
where the alkyl and aryl groups mentioned may be substituted;
X¹² is selected from:
tert-butyl, -methylthioethyl, -trimethylsilyl, -triethysilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl, tetrahydropyranyl, -allyl ether, -benzyl, -p-methoxybenzyl, -3,4-dimethoxybenzyl, -aryl, -methoxymethyl.

Process for preparing 2,2′-dihydroxybiaryls, comprising the process steps of:

a3) reacting a compound of the formula (Xa):

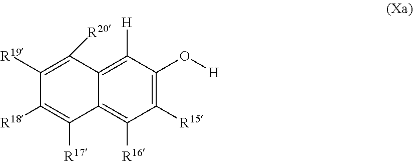

with X^{12′} to give (Xb):

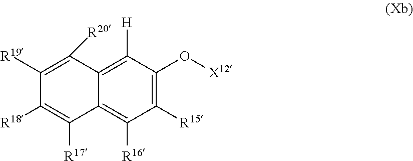

b3) electrochemically coupling;

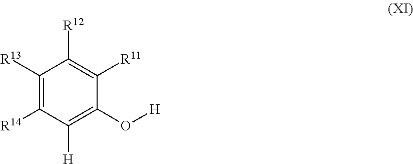

(Xb) with (XI) to give (XII)
with use of the compound having the higher oxidation potential in excess:

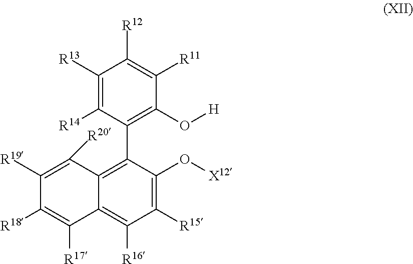

where
R¹¹, R¹², R¹³, R¹⁴ are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —CN, —N[(C₁-C₁₂)-alkyl]₂:
R^{15′}, R^{16′}, R^{17′}, R^{18′}, R^{19′}, R^{20′} are selected from:
—H, —(C₁-C₁₂)-alkyl, —O—(C₁-C₁₂)alkyl, —O—(C₆-C₂₀)-aryl, —(C₆-C₂₀)aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —N[(C₁-C₁₂)-alkyl]₂;
where the alkyl and aryl groups mentioned may be substituted;
X^{12′} is selected from:
tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl, tetrahydropyranyl, -allyl ether, -benzyl, -p-methoxybenzyl, -3,4-dimethoxybenzyl, -aryl, -methoxymethyl.

Process for preparing 2,2′-dihydroxybiaryls, comprising the process steps of:

a4) reacting a compound of the formula (XIIIa):

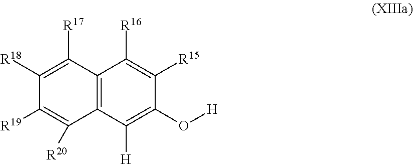

with X¹³ to give (XIIIb):

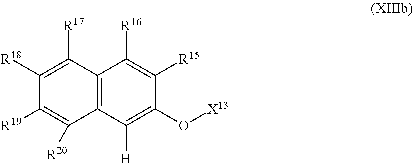

b4) electrochemically coupling:

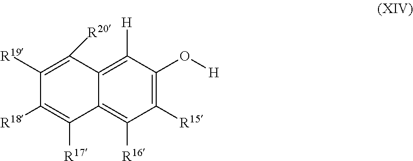

(XIIIb) with (XIV) to give (XV)
with use of the compound having the higher oxidation potential in excess:

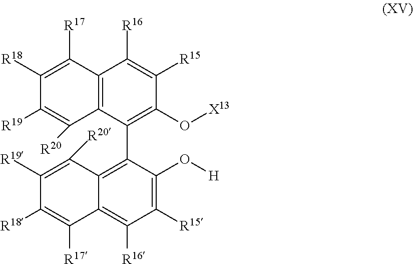

where
R¹⁵, R¹⁶, R¹⁷, R¹⁸, R¹⁹, R²⁰, R^{15′}, R^{16′}, R^{17′}, R^{18′}, R^{19′}, R^{20′} are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —N[(C₁-C₁₂)-alkyl]₂;
where the alkyl and aryl groups mentioned may be substituted;
X¹³ is selected from:
tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl, tetrahydropyranyl, -allyl ether, -benzyl, -p-methoxybenzyl, -3,4-dimethoxybenzyl, -aryl, -methoxymethyl.

The process variants cited hereinafter relate to all four aforementioned processes, with the proviso that the radicals specified in the variant occur in the process.

In one variant of the process, X¹¹ is selected from:

-tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl.

In one variant of the process, X¹² is selected from:

tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl.

In one variant of the process, X^{12′} is selected from:

tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl.

In one variant of the process, X¹³ is selected from:

tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl.

In one variant of the process, X¹¹ is selected from:

-tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester.

In one variant of the process, X¹² is selected from:

-tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester.

In one variant of the process, X^{12′} is selected from:

-tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester.

In one variant of the process, X¹³ is selected from:

-tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester.

In one variant of the process, X¹¹ is selected from:

-tert-butyl, -methylthioethyl, -trimethylsilyl, -triethylsilyl, -triisopropylsilyl, -tert-butyldimethylsilyl, -tert-butyldiphenylsilyl, -benzoic ester.

In one variant of the process, X¹² is selected from:

-tert-butyl, -methylthioethyl, -trimethylsilyl, -triethylsilyl, -triisopropylsilyl, -tert-butyldimethylsilyl, -tert-butyldiphenylsilyl, -benzoic ester.

In one variant of the process, X^{12′} is selected from:

-tert-butyl, -methylthioethyl, -trimethylsilyl, -triethylsilyl, -triisopropylsilyl, -tert-butyldimethylsilyl, -tert-butyldiphenylsilyl, -benzoic ester.

In one variant of the process, X¹³ is selected from:

-tert-butyl, -methylthioethyl, -trimethylsilyl, -triethylsilyl, -triisopropylsilyl, -tert-butyldimethylsilyl, -tert-butyldiphenylsilyl, -benzoic ester.

In one variant of the process, X¹¹ is selected from:

-trimethylsilyl, -triethylsilyl, -triisopropylsilyl, -tert-butyldimethylsilyl, -tert-butyldiphenylsilyl.

In one variant of the process, X¹² is selected from:

-trimethylsilyl, -triethylsilyl, -triisopropylsilyl, -tert-butyldimethylsilyl, -tert-butyldiphenylsilyl.

In one variant of the process, X^{12′} is selected from:

-trimethylsilyl, -triethylsilyl, -triisopropylsilyl, -tert-butyldimethylsilyl, -tert-butyldiphenylsilyl.

In one variant of the process, X¹³ is selected from:

-trimethylsilyl, -triethylsilyl, -triisopropylsilyl, -tert-butyldimethylsilyl, -tert-butyldiphenylsilyl.

In one variant of the process, X¹¹ is selected from:

tert-butyl, -methylthioethyl, -acetyl, -pivaloyl, -benzoic ester.

In one variant of the process, X¹² is selected from:

tert-butyl, -methylthioethyl, -acetyl, -pivaloyl, -benzoic ester.

In one variant of the process, X^{12′} is selected from:

tert-butyl, -methylthioethyl, -acetyl, -pivaloyl, -benzoic ester.

In one variant of the process, X¹³ is selected from:

tert-butyl, -methylthioethyl, -acetyl, -pivaloyl, -benzoic ester.

In one variant of the process, X¹¹ is selected from:

tert-butyl, -acetyl, -pivaloyl, -benzoic ester.

In one variant of the process, X¹² is selected from:

tert-butyl, -acetyl, -pivaloyl, -benzoic ester.

In one variant of the process, X^{12′} is selected from:

tert-butyl, -acetyl, -pivaloyl, -benzoic ester.

In one variant of the process, X¹³ is selected from:

tert-butyl, -acetyl, -pivaloyl, -benzoic ester.

In one variant of the process, X¹¹ is selected from:

-acetyl, -pivaloyl.

In one variant of the process, X¹² is selected from:

-acetyl, -pivaloyl.

In one variant of the process, X^{12′} is selected from:

-acetyl, -pivaloyl.

In one variant of the process, X¹³ is selected from:

-acetyl, -pivaloyl.

In one variant of the process, R¹¹, R¹², R¹³, R¹⁴, R^{11′}, R^{12′}, R^{13′}, R^{14′} are selected from:

—H, —(C₁-C₁₂)-alkyl, —O—(C₁-C₁₂)-alkyl, —O—(C₆-C₂₀)-aryl, —S-alkyl, —S-aryl, halogen.

In one variant of the process, R¹¹, R¹², R¹³, R¹⁴, R^{11′}, R^{12′}, R^{13′}, R^{14′} are selected from:

—H, —(C₁-C₁₂)-alkyl, —O—(C₁-C₁₂)-alkyl, —O—(C₆-C₂₀)-aryl.

In one variant of the process, R^{15′}, R^{16′}, R^{17′}, R^{18′}, R^{19′}, R^{20′} are selected from:

—H, —(C₁-C₁₂)-alkyl, —O—(C₁-C₁₂)-alkyl, —O—(C₆-C₂₀)-aryl, —S-alkyl, —S-aryl, halogen.

In one variant of the process, R^{15′}, R^{16′}, R^{17′}, R^{18′}, R^{19′}, R^{20′} are selected from:

—H, —(C₁-C₁₂)-alkyl, —O—(C₁-C₁₂)-alkyl, —O—(C₆-C₂₀)-aryl.

In one variant of the process, R¹⁵, R¹⁶, R¹⁷, R¹⁸, R¹⁹, R²⁰ are selected from:

—H, —(C₁-C₁₂)-alkyl, —O—(C₁-C₁₂)-alkyl, —O—(C₆-C₂₀)-aryl, —S-alkyl, —S-aryl, halogen.

In one variant of the process, R¹⁵, R¹⁶, R¹⁷, R¹⁸, R¹⁹, R²⁰ are selected from:

—H, —(C₁-C₁₂)-alkyl, —O—(C₁-C₁₂)-alkyl, —O—(C₆-C₂₀)-aryl.

In one variant, the two radicals in at least one of the four following radical pairs are not the same radical: R¹¹ and R^{11′}, R¹² and R^{12′}, R¹³ and R^{13′}, R¹⁴ and R^{14′}.

In one variant, the two radicals in at least one of the six following radical pairs are not the same radical: R¹⁵ and R^{15′}, R¹⁶ and R^{16′}, R¹⁷ and R^{17′}, R¹⁸ and R^{18′}, R¹⁹ and R^{19′}, R²⁰ and R^{20′}.

The feature “and the two radicals in at least one of the four following radical pairs are not the same radical: R¹¹ and R^{11′}, R¹² and R^{12′}, R¹³ and R^{13′}, R¹⁴ and R^{14′}” expresses the fact that this is an unsymmetric biaryl. The two aromatic systems cannot be reflected onto one another by a mirror plane lying between them. Not even if X¹¹ were to be H.

The following radical pairs are permitted, for example:

R¹¹≠R^{11′}, R¹²=R^{12′}, R¹³=R^{13′}, R¹⁴=R^{14′};
R¹¹=R^{11′}, R¹²=R^{12′}, R¹³≠R^{13′}, R¹⁴=R^{14′}.

But also radical pairs in which more than just one pair is not the same, for example:

R¹¹≠R^{11′}, R¹²=R^{12′}, R¹³≠R^{13′}, R¹⁴=R^{14′};
R¹¹≠R^{11′}, R¹²≠R^{12′}, R¹³≠R^{13′}, R¹⁴=R^{14′}.

The only case ruled out is that in which all four radical pairs are each the same radical in pairs:

R¹¹=R^{11′}, R¹²=R^{12′}, R¹³=R^{13′}, R¹⁴=R^{14′}.

This would be a symmetric biaryl.

The same applies to the following pairs: R¹⁵ and R^{15′}, R¹⁶ and R^{16′}, R¹⁷ and R^{17′}, R¹⁸ and R^{18′}, R¹⁹ and R^{19′}, R²⁰ and R^{20′}.

By electrochemical coupling (process step b)), biaryls are prepared without having to add organic oxidizing agents, work with exclusion of moisture or maintain anaerobic reaction conditions. This direct method of C,C coupling opens up an inexpensive and environmentally beneficial alternative to existing multistage conventionally organic synthesis routes.

Process step b) can be conducted using different carbon electrodes (glassy carbon, boron-doped diamond, graphite, carbon fibres, nanotubes, inter alia), metal oxide electrodes and metal electrodes. This involves applying current densities in the range of 1-50 mA/cm².

The electrochemical coupling (process step b)) is conducted in the customary, known electrolysis cells.

In one variant of the process, the compound having the higher oxidation potential is used in at least twice the amount compared to the compound having the lower oxidation potential.

In one variant of the process, the ratio of the compound having the lower oxidation potential to the compound having the higher oxidation potential is in the range from 1:2 to 1:4.

If required, a conductive salt can be added to the reaction.

In one variant of the process, the conductive salt is selected from the group of the alkali metal, alkaline earth metal, tetra(C₁-C₆-alkyl)ammonium, 1,3-di(C₁-C₆-alkyl)imidazolium and tetra(C₁-C₆-alkyl)phosphonium salts.

In one variant of the process, the counterions of the conductive salts are selected from the group of sulphate, hydrogensulphate, alkylsulphates, arylsulphates, alkylsulphonates, arylsulphonates, halides, phosphates, carbonates, alkylphosphates, alkylcarbonates, nitrate, tetrafluoroborate, hexafluorophosphate, hexafluorosilicate, fluoride and perchlorate.

In one variant of the process, the conductive salt is selected from tetra-(C₁-C₆-alkyl)ammonium salts, and the counterion from sulphate, alkylsulphate, arylsulphate.

The workup and recovery of the biaryls is very simple and is effected, after the reaction has ended, by generally standard separation methods. First of all, the electrolyte solution is removed by means of distillation and the individual compounds are obtained separately in the form of different fractions. A further purification can be effected, for example, by crystallization, distillation, sublimation or chromatography.

A problem that occurs in the electrochemical coupling of different molecules is that the co-reactants generally have different oxidation potentials E_{Ox}. The result of this is that the molecule having the lower oxidation potential has a higher propensity to release an electron (e⁻) to the anode and an H⁺ ion to the solvent, for example, than the molecule having the lower oxidation potential. The oxidation potential E_{Ox} can be calculated via the Nernst equation:

E_{Ox}=E°+(0.059/n)*Ig([Ox]/[Red])

E_{Ox}: Electrode potential for the oxidation reaction (=oxidation potential)
E°: Standard electrode potential
n: Number of electrons transferred
[Ox]: Concentration of the oxidized form
[Red]: Concentration of the reduced form

If a process known from the coupling of two identical aryls were to be applied to two different aryls, this would result in predominant formation of radicals of the molecule having a lower oxidation potential, and these would then react with themselves. By far the predominant main product obtained would thus be a biaryl which has formed from two identical aryls.

The oxidation potentials of the respective phenol and/or naphthol derivatives depend both on the protecting group used in each case and on the structure of the substrate itself. According to the protecting group used, a change in the oxidation potential by several hundred millivolts is possible. This adjustment of the oxidation potentials is possible via electron-withdrawing or electron-donating groups, but also via different sizes and the associated steric effects. The process according to the invention thus opens up an additional means of controlling the oxidation potential of the phenol and naphthol derivatives via the protecting groups.

In addition, it is possible to shift the oxidation potentials of the substrates used through the controlled addition of protic additives such as methanol or water to the electrolyte (such as HFIP: 1,1,1,3,3,3-hexafluoro-2-propanol).

Through electrochemical treatment of phenols/naphthols with O-protected derivatives, unsymmetric, partly protected 2,2′-dihydroxybiaryls are prepared without having to add oxidizing agents, work with exclusion of moisture or observe anaerobic reaction regimes. The specific choice of the protecting groups and the choice of the solvent enable the control of the oxidation potentials of the substrates used and thus indicate the success of a C,C cross-coupling reaction. The obtainability of only one hydroxyl function in such biaryls has been enabled to date only through the unselective subsequent introduction of a protecting group in a two-stage synthesis.

In the process according to the invention, in contrast, it is also possible to tolerate tert-butyl groups and other electrophilically labile R₁ and R₂ radicals. This opens up the route to introduction of functions at the free OH group which would not be reconcilable with the oxidative cross-coupling.

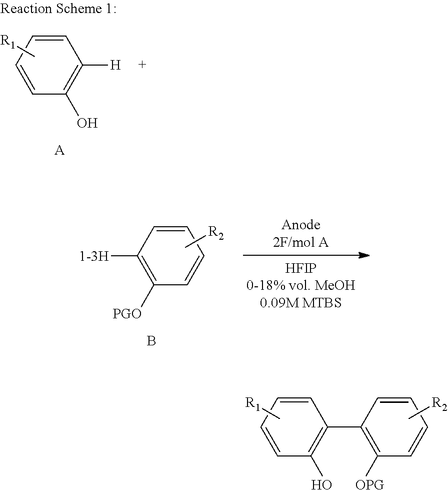

Reaction Scheme 1: Electrochemical synthesis of unsymmetric, partly protected biphenols
HFIP: 1,1,1,3,3,3-hexafluoro-2-propanol
MTBS: Bu₃NMe MeOSO₃
PG: protecting group

The reaction scheme applies analogously to the coupling of a phenol to a naphthol or of two naphthols to one another.

The invention is illustrated in detail hereinafter by working examples and figures.

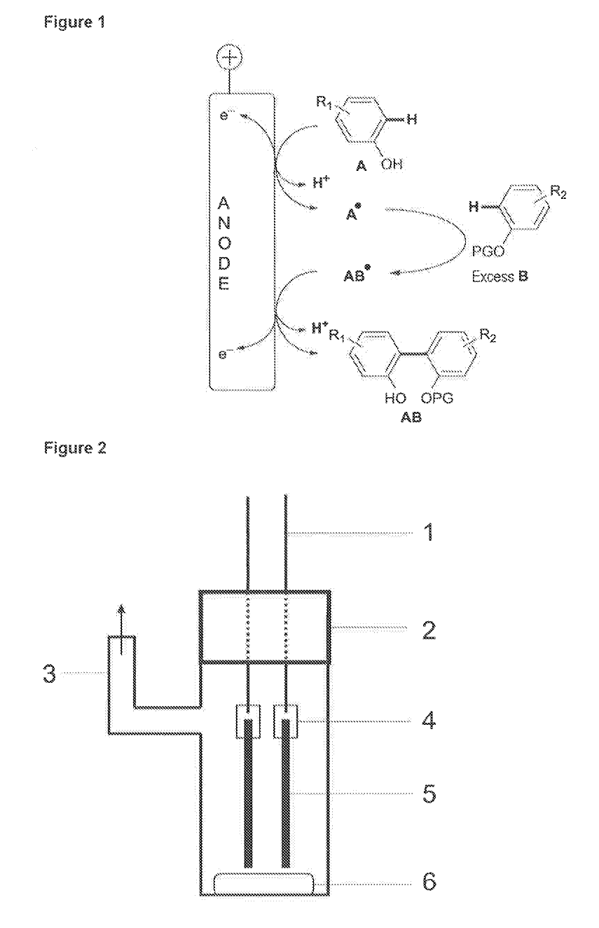

FIG. 1 shows, in schematic form, the electrochemical processes at the anode. This shows the mechanical concept for electrochemical formation of unsymmetric, partly protected dihydroxybiaryl derivatives.

There is selective oxidation of the phenol component A, which has a lower oxidation potential than B. As a result of the high reactivity of the free-radical species A. formed, the latter is capable of being attacked nucleophilically by component B. The first oxidation potentials of the two substances are crucial to the course of the reaction. The controlled addition of protic additives such as MeOH or water to the electrolyte can enable a shift in precisely these oxidation potentials. Thus, yield and selectivity of the reaction become controllable.

(PG: protecting group)

FIG. 2 shows the schematic setup of a reaction apparatus in which the coupling reaction to give the corresponding unsymmetric 2,2′-biaryls can be conducted. The reaction apparatus comprises glassy carbon electrodes (5) held with stainless steel clamps (4). A magnetic stirrer bar (6) ensures mixing in the reaction apparatus. A Teflon stopper (2) rests on top of the reaction apparatus, through which stainless steel holders (1) for the electrodes lead. The reaction apparatus, a beaker cell here, has a fitted outlet (3) for a reflux condenser attachment.

Examples of possible protecting groups:

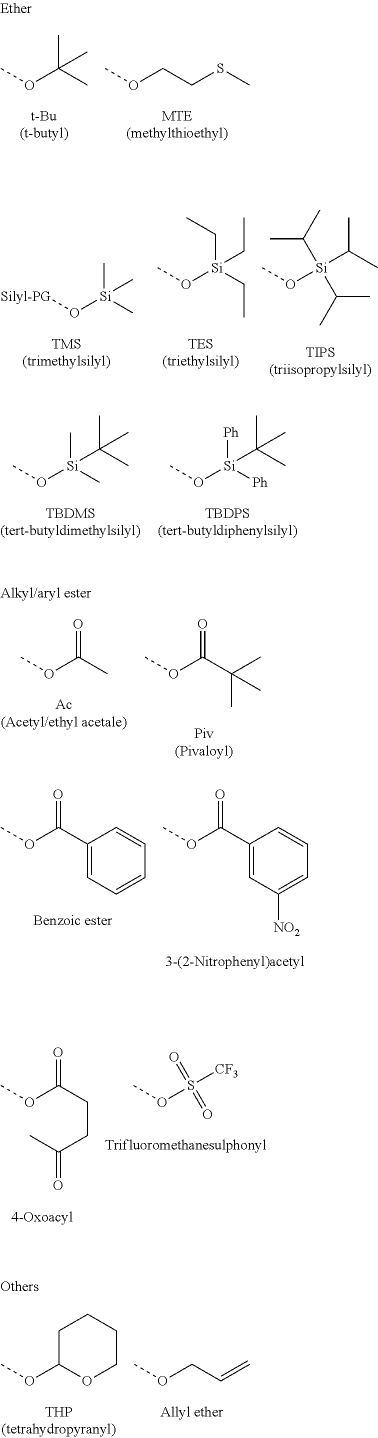

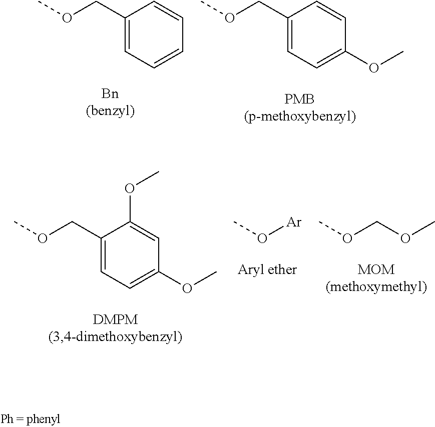

The introduction of the protecting groups can be effected, for example, as described in P. G. M. Wuts, T. W. Greene “Greene's Protective Groups in Organic Synthesis”, fourth edition, 2007, John Wiley and Sons; Hoboken, N.J.

The process according to the invention does not have the disadvantages mentioned in the prior art (A. Kirste, B. Elsler, G. Schnakenburg, S. R. Waldvogel, in J. Am. Chem. Soc., 2012, 134, 3571-3576). By virtue of the protecting group already being introduced in the first process step, costly purification steps at the end of the synthesis chain which lead to minimization of product of value are avoided. An additional factor is that the protecting group can be introduced selectively into one phenol. No reaction of any other OH group takes place, since the compound contains only one OH group, in contrast to the later dihydroxybiaryl.

1. Compound having one of the general structures (I) to (IIb): 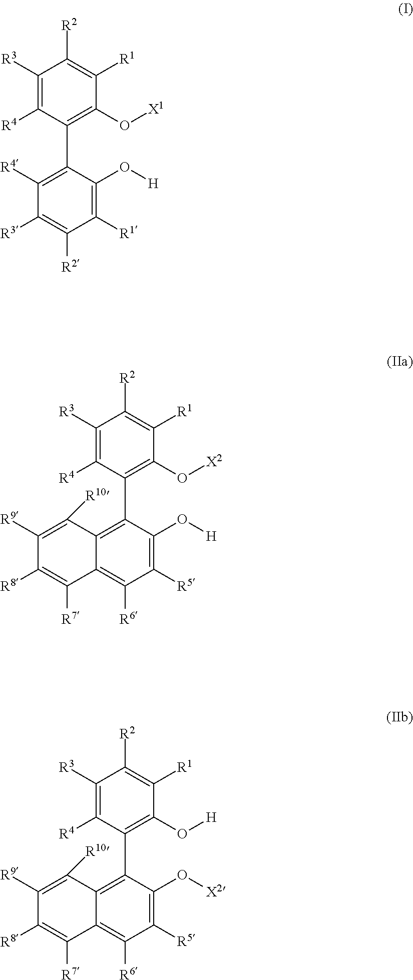
where
R¹, R², R³, R⁴, R^{1′}, R^{2′}, R^{3′}, R^{4′} are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —CN, —N[(C₁-C₁₂)-alkyl]₂;
R^{5′}, R^{6′}, R^{7′}, R^{8′}, R^{9′}, R^{10′} are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —N[(C₁-C₁₂)-alkyl]₂;
where the alkyl and aryl groups mentioned may be substituted;
and, in the formula (I), the two radicals in at least one of the four following radical pairs are not the same radical: R¹ and R^{1′}, R² and R^{2′}, R³ and R^{3′}, R⁴ and R^{4′},
X² is selected from:
tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl,
X^{2′} is selected from:
tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl;
X¹ is selected from:
-trimethylsilyl, -triethylsilyl, -triisopropylsilyl, -tert-butyldimethylsilyl, -tert-butyldiphenylsilyl.

2. Compound according to claim 1,
where R¹, R², R³, R⁴, R^{1′}, R^{2′}, R^{3′} are selected from:
—H, —(C₁-C₁₂)-alkyl, —O—(C₁-C₁₂)-alkyl, —O—(C₆-C₂₀)-aryl, —S-alkyl, —S-aryl, halogen.

3. Compound according to claim 1,
where R^{5′}, R^{6′}, R^{7′}, R^{8′}, R^{9′}, R^{10′} are selected from:
—H, —(C₁-C₁₂)-alkyl, —O—(C₁-C₁₂)-alkyl, —O—(C₆-C₂₀)-aryl, —S-alkyl, —S-aryl, halogen.

4. Compound according to claim 1,
where the compound has the general structure (I).

5. Compound according to claim 1,
where the compound has the general structure (IIa) or (IIb).

6. Process for preparing 2,2′-dihydroxybiaryls, comprising the process steps of:
a1) reacting a compound of the formula (IVa): 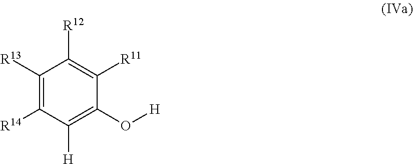
with X¹¹ to give (IVb) 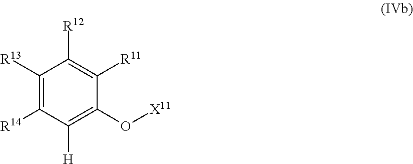
b1) electrochemically coupling: 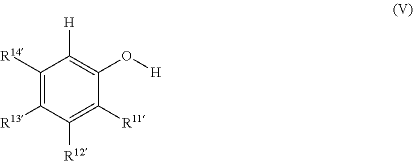
(IVb) with (V) to give (VI)
with use of the compound having the higher oxidation potential in excess: 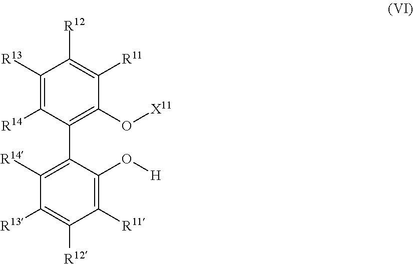
where
R¹¹, R¹², R¹³, R¹⁴, R^{11′}, R^{12′}, R^{13′}, R^{14′} are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —CN, —N[(C₁-C₁₂)-alkyl]₂;
where the alkyl and aryl groups mentioned may be substituted;
X¹¹ is selected from:
tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl, tetrahydropyranyl, -allyl ether, -benzyl, -p-methoxybenzyl, -3,4-dimethoxybenzyl, -aryl, -methoxymethyl.

7. Process for preparing 2,2′-dihydroxybiaryls, comprising the process steps of:
a2) reacting a compound of the formula (VIIa): 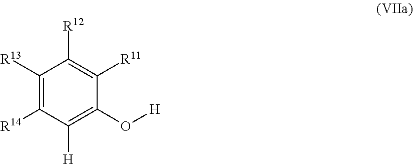
with X¹² to give (VIIb): 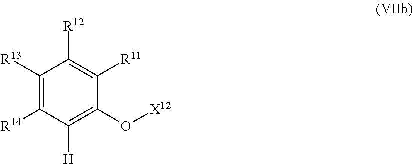
b2) electrochemically coupling: 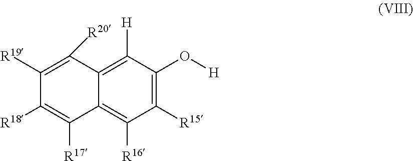
(VIIb) with (VIII) to give (IX)
with use of the compound having the higher oxidation potential in excess: 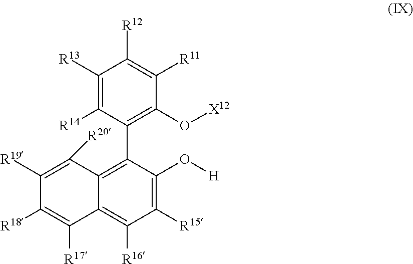
where
R¹¹, R¹², R¹³, R¹⁴ are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —CN, —N[(C₁-C₁₂)-alkyl]₂;
R^{15′}, R^{16′}, R^{17′}, R^{18′}, R^{19′}, R^{20′} are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —N[(C₁-C₁₂)-alkyl]₂;
where the alkyl and aryl groups mentioned may be substituted;
X¹² is selected from:
tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl, tetrahydropyranyl, -allyl ether, -benzyl, -p-methoxybenzyl, -3,4-dimethoxybenzyl, -aryl, -methoxymethyl.

8. Process for preparing 2,2′-dihydroxybiaryls, comprising the process steps of:
a3) reacting a compound of the formula (Xa): 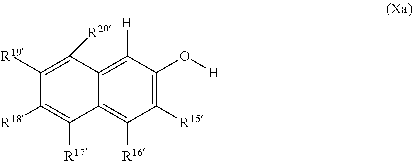
with X^{12′} to give (Xb); 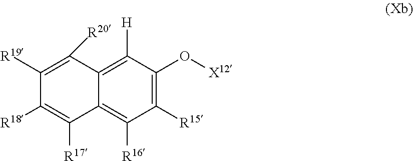
b3) electrochemically coupling: 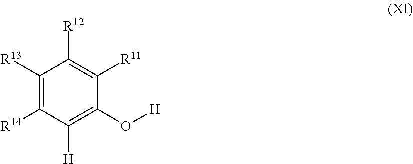
(Xb) with (XI) to give (XII)
with use of the compound having the higher oxidation potential in excess: 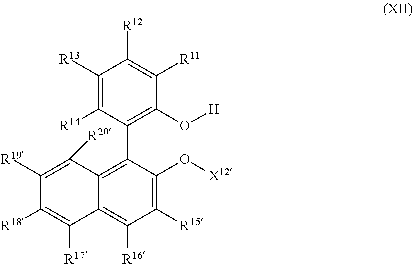
where
R¹¹, R¹², R¹³, R¹⁴ are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —CN, —N[(C₁-C₁₂)-alkyl]₂;
R^{15′}, R^{16′}, R^{17′}, R^{18′}, R^{19′}, R^{20′} are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —N[(C₁-C₁₂)-alkyl]₂;
where the alkyl and aryl groups mentioned may be substituted;
X^{12′} is selected from:
tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl, tetrahydropyranyl, -allyl ether, -benzyl, -p-methoxybenzyl, -3,4-dimethoxybenzyl, -aryl, -methoxymethyl.

9. Process for preparing 2,2′-dihydroxybiaryls, comprising the process steps of:
a4) reacting a compound of the formula (XIIIa): 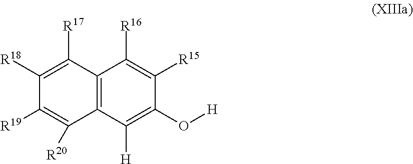
with X¹³ to give (XIIIb): 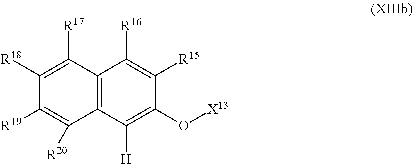
b4) electrochemically coupling: 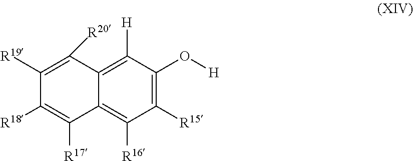
(XIIIb) with (XIV) to give (XV)
with use of the compound having the higher oxidation potential in excess: 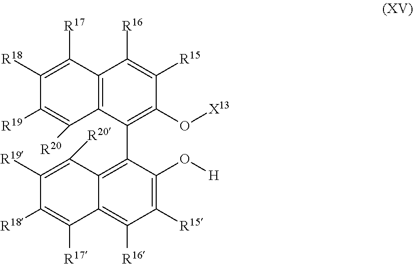
where
R¹⁵, R¹⁶, R¹⁷, R¹⁸, R¹⁹, R²⁰, R^{15′}, R^{16′}, R^{17′}, R^{18′}, R^{19′}, R^{20′} are selected from:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —SO₃H, —N[(C₁-C₁₂)-alkyl]₂;
where the alkyl and aryl groups mentioned may be substituted;
X¹³ is selected from:
tert-butyl, -methylthioethyl, -trimethylsilyl, -triethylsilyl, -triisopropylsilyl, -tert-butyldimethylsilyl, -tert-butyldiphenylsilyl, -acetyl, -pivaloyl, -benzoic ester, -3-(2-nitrophenyl)acetyl, -oxoacyl, -trifluoromethanesulphonyl, tetrahydropyranyl, -allyl ether, -benzyl, -p-methoxybenzyl, -3,4-dimethoxybenzyl, -aryl, -methoxymethyl.

10. (canceled)
